(19) United States
(12) Patent Application Publication
(10) Pub. No.: US 2022/0391711 A1
(43) Pub. Date: Dec. 8, 2022

(54) DYNAMIC SCHEDULING SYSTEM AND METHOD OF DYEING PROCESS USING GENETIC ALGORITHM

(71) Applicant: ELECTRONICS AND TELECOMMUNICATIONS RESEARCH INSTITUTE, Daejeon (KR)

(72) Inventors: Hyun Woo OH, Daejeon (KR); Young Seog YOON, Daejeon (KR)

(21) Appl. No.: 17/665,345

(22) Filed: Feb. 4, 2022

(30) Foreign Application Priority Data
May 27, 2021 (KR) 10-2021-0068367

(51) Int. Cl. G06N 3/12; G05B 19/418; D06B 23/00

(52) U.S. Cl. CPC G06N 3/126; G05B 19/41865; D06B 23/00

(57) ABSTRACT

The present invention relates to a dynamic scheduling system and method of a dyeing process using a genetic algorithm, and more particularly, to technology which performs an optimized process corresponding to an ordered work command in a dyeing process by using process scheduling based on a genetic algorithm to increase production efficiency.

### CROSS-REFERENCE TO RELATED APPLICATIONS

This application claims priority under 35 U.S.C. § 119 to Korean Patent Application No. 10-2021-0068367, filed on May 27, 2021, the disclosure of which is incorporated herein by reference in its entirety.

### TECHNICAL FIELD

The present invention relates to a dynamic scheduling system and method of a dyeing process using a genetic algorithm, and more particularly, to technology which performs an optimized process corresponding to an ordered work command in a dyeing process by using process scheduling based on a genetic algorithm to increase production efficiency.

### BACKGROUND

Fourth Industrial Revolution, attracting much attention recently, may be considered to start in the smart manufacturing field. It is considered that the fiber field including fabric manufacturing, dyeing, processing, shoes, and clothes is included in one of fields where information and communication technology associated with smart manufacturing is not normally introduced. In detail, many companies associated with manufacturing are operating manual control facilities globally and some automated facilities have been introduced, but it may be considered that information and communication technology is not used or merged yet.

In a process of such manufacturing field, scheduling based on various factors such as situations of secured raw materials and capacities of facilities should be planned even when many works are allocated, and it is very important to schedule a process capable of being performed despite an accidental situation such as receiving a work command where a due date is urgent. For example, a manager directly plays a role in and processes process scheduling based on a production plan in a current dyeing process. Here, scheduling of a dyeing process may be performed based on various factors such as allocation of a dyeing machine and a capacity of the dyeing machine based on the kind of fabric to be dyed, the kind, color, and concentration of dyeing, and the continuity of a process, and as described above, when the number of factors to be considered increases, a difference in production efficiency of each process may occur based on the capability of a manager processing scheduling. Also, a case where scheduling is again performed occurs based on situations of a site where a process is performed, or a case, where it is needed to change scheduling due to an urgently-requested work command, occurs frequently. Due to this, there is a problem where a manager should always reside at a site.

### SUMMARY

Accordingly, the present invention provides a dynamic scheduling system and method of a dyeing process using a genetic algorithm, in which process scheduling, which satisfies the product quality and due date condition of a dyeing process even without depending on a process scheduling manager and maximizes production efficiency on the basis of various factors, is made in planning work scheduling corresponding to an ordered work command in the dyeing process.

In one general aspect, a dynamic scheduling method of a dyeing process using a genetic algorithm, which optimizes performing of an ordered work command through scheduling on each process of the dyeing process, includes: selecting a work command, which is to be performed, from among ordered work command in the dyeing process; setting each process, needed for performing the selected work command, to a unit gene and combining unit genes, set to correspond to each process, to generate an initial chromosome; allocating a machine for performing a dyeing process on each unit gene configuring the initial chromosome; and calculating suitability for the initial chromosome to which the machine is allocated.

In another general aspect, a dynamic scheduling system for a dyeing process using a genetic algorithm, which optimizes performing of an ordered work command through scheduling on each process of the dyeing process, includes: a data collector configured to collect order data associated with the ordered work command and process data associated with each process included in a dyeing process; a work time calculator configured to calculate a work time of each process on the basis of the collected order data and process data; a work command determiner configured to select a work command, which is to be performed, from among ordered work commands on the basis of the calculated work time; a chromosome generator configured to generate initial chromosomes, set to unit genes for configuring a chromosome in each process needed for performing the selected work command, and a dyeing machine-allocation chromosome for allocating a dyeing machine for unit chromosomes corresponding to a sub dyeing process of each process; and a process controller configured to perform each process on the basis of a configuration of a chromosome representing optimal suitability among the generated initial chromosomes to perform the selected work command.

In another general aspect, a dynamic scheduling system for a manufacturing process of optimizing performing of an ordered work command through scheduling on each process of the manufacturing process includes: a data collector configured to collect order data associated with an ordered work command in the manufacturing process and process data associated with each process included in the manufacturing process; a work time calculator configured to calculate a work time of each process on the basis of the collected order data and process data; a work command determiner configured to select a work command, which is to be performed, from among ordered work commands on the basis of the calculated work time; a chromosome generator configured to set each process, needed for performing the selected work command, to unit genes and combine the unit genes, set to correspond to each process, to generate a chromosome having optimal suitability for the manufacturing process; and a process controller configured to perform each process on the basis of a configuration of a chromosome representing optimal suitability among the generated initial chromosomes to perform the selected work command.

Other features and aspects will be apparent from the following detailed description, the drawings, and the claims.

### BRIEF DESCRIPTION OF THE DRAWINGS

FIG. 1 is a block diagram illustrating a configuration of a dynamic scheduling system of a dyeing process using a genetic algorithm according to the present invention.

FIG. 2 is a reference diagram for describing an initial chromosome generating process of a dynamic scheduling system of a dyeing process using a genetic algorithm according to an embodiment of the present invention.

FIGS. 3 to 5 are reference diagrams for describing an initial chromosome configuration and an initial chromosome generation generating process of a dynamic scheduling system of a dyeing process using a genetic algorithm according to an embodiment of the present invention.

FIG. 6A and 6C are reference diagram for describing a machine allocation process for a sub dyeing process and other processes included in a dyeing process of a dynamic scheduling system of a dyeing process using a genetic algorithm according to an embodiment of the present invention.

FIGS. 7 to 8B are reference diagrams for describing a machine allocation process for a dyeing process of a dynamic scheduling system of a dyeing process using a genetic algorithm according to an embodiment of the present invention.

### DETAILED DESCRIPTION OF EMBODIMENTS

Embodiments of the present invention are provided so that this disclosure will be thorough and complete, and will fully convey the concept of the present invention to one of ordinary skill in the art. Since the present invention may have diverse modified embodiments, preferred embodiments are illustrated in the drawings and are described in the detailed description of the present invention. However, this does not limit the present invention within specific embodiments and it should be understood that the present invention covers all the modifications, equivalents, and replacements within the idea and technical scope of the present invention. In describing the present invention, a detailed description of known techniques associated with the present invention unnecessarily obscure the gist of the present invention, it is determined that the detailed description thereof will be omitted.

Moreover, each of terms such as “. . . part”, “. . . unit”, and “module” described in specification denotes an element for performing at least one function or operation, and may be implemented in hardware, software or the combination of hardware and software.

In the following description, the technical terms are used only for explain a specific exemplary embodiment while not limiting the present invention. The terms of a singular form may include plural forms unless referred to the contrary. The meaning of ‘comprise’, ‘include’, or ‘have’ specifies a property, a region, a fixed number, a step, a process, an element and/or a component but does not exclude other properties, regions, fixed numbers, steps, processes, elements and/or components.

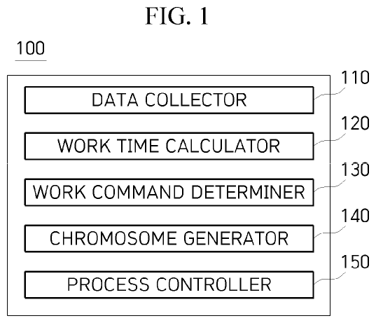

Referring to FIG. 1, a dynamic scheduling system 100 of a dyeing process using a genetic algorithm (hereinafter referred to as a dynamic scheduling system) according to the present invention may be for optimizing performing of an ordered word command through scheduling of each process of a dyeing process and may include a data collector 110, a work time calculator 120, a work command determiner 130, a chromosome generator 140, and a process controller 150.

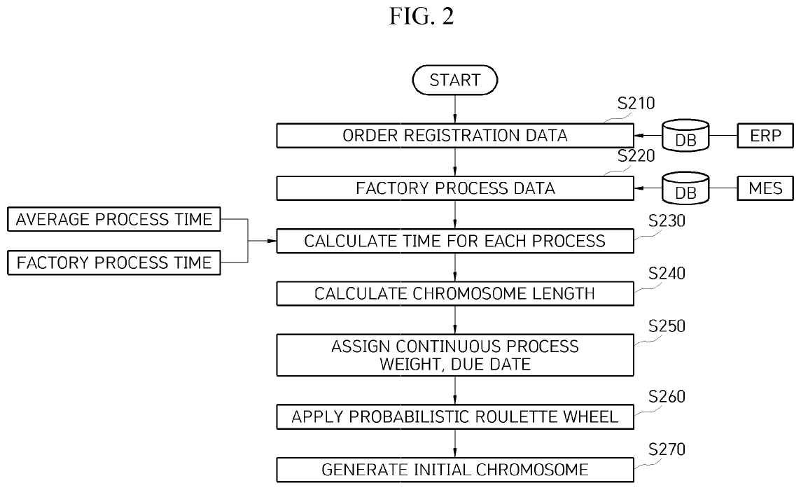

FIG. 2 is a reference diagram for describing an initial chromosome generating process of a dynamic scheduling system of a dyeing process using a genetic algorithm according to an embodiment of the present invention.

Referring to FIG. 2, the data collector 110 may collect order data associated with an ordered work command and process data associated with each process included in a dyeing process in steps S210 and S220. In detail, the order data associated with the ordered work command in the dyeing process may include detailed information about the ordered work command like a company of a customer, the kind of a raw material, a name of the raw material, a dyeing color, processing information, and a special situation and may be collected by using an enterprise resource planning (ERP) system. Also, the process data may include information for checking which work is performed in each process included in the dyeing process. It may be understood that “process” other than the dyeing process described herein is a small-unit process configuring the dyeing process generally.

Subsequently, in step S230, the work time calculator 120 may calculate a work time of each process on the basis of the collected order data and process data in step S230. Each of the dyeing process may have an average work time, but the work time calculator 120 may predict an estimation work time and a work time which vary based on a characteristic of each process, on the basis of the collected order data and process data.

Subsequently, the work command determiner 130 may select a work command, which is to be performed, from among ordered work commands on the basis of the calculated work time. In detail, the dyeing process may have a limitation in capability to process a work command, and thus, may select a work command, where process scheduling is to be performed, from among all ordered work commands on the basis of a unit period such as a day or may select a work command where process scheduling is to be performed, on the basis of a predetermined production plan, thereby using a method which continuously performs a work command equal to a possibility of accommodation.

Subsequently, the chromosome generator 140 may set each process, needed for performing the selected work command, to a unit chromosome and may combine unit chromosomes set to correspond to each process, thereby generating a chromosome having optimal suitability for the dyeing process. Also, the chromosome generator 140 may generate dyeing machine-allocation chromosome for allocating a dyeing machine for unit chromosomes corresponding to a sub dyeing process of each process. Here, each process set to the unit chromosome may include, for example, a plurality of processes such as a coloring process, a preprocessing process, a sub dyeing process, and an atmospheric pressure process, and a series of a process may be changed based on situations of each dyeing process.

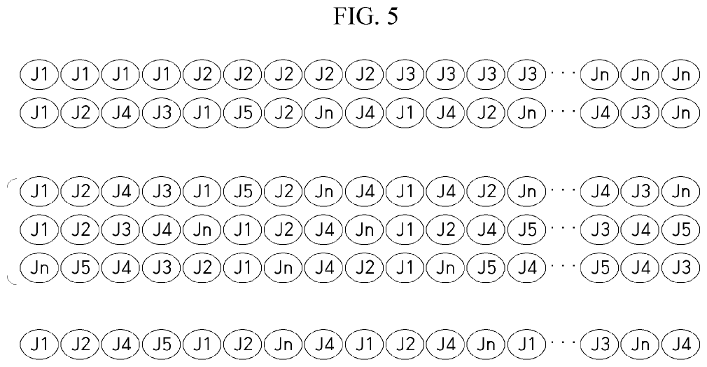

Moreover, in step S240, the chromosome generator 140 may calculate a length of a chromosome configured by a combination of unit genes. Here, the length of the chromosome may denote a value which is a sum of unit genes, and the chromosome generator 140 may determine, as a length of a chromosome, a value which is a sum of unit genes (processes) configuring each of the selected work commands. Referring to FIGS. 4 and 5, for example, J1 to Jn in FIGS. 4 and 5 may each denote a work command, and M1 to Mn may each denote a process needed for each work command. In FIG. 4, the total number of work commands selected for performing process scheduling in a dyeing process may be an n number (J1 to Jn), a process needed for J1 may include four processes (M1, M2, M3, and M4), a process needed for J2 may include five processes (M1, M2, M3, M4, and M5), and a process needed for J3 may include four processes (M1, M4, M5, and M6). In this case, like chromosomes shown in a first row of FIG. 5, a chromosome including unit genes may be generated. Also, in the first row of FIG. 5, an example where chromosomes are generated by arranging unit genes is shown, and when suitability is calculated after chromosomes are generated by the number of cases which are obtained by applying crossover and mutation to chromosomes generated by such a method, a very long time may be taken in completing process scheduling. Therefore, initial chromosomes may be generated by implementing efficient unit genes arrangement by using a below-described method, and then, suitability for the generated initial chromosomes may be calculated and process scheduling may be quickly completed. Furthermore, in the chromosomes of the first row of FIG. 5, arrangement of unit genes and machine allocation for a dyeing process are not completed, and when an operation of arranging the unit genes and an operation of allocating a machine for the dyeing process are completed by a below-described process, it may be considered that process scheduling using a genetic algorithm according to the present invention is completed.

Moreover, the chromosome generator 140 may implement the unit genes arrangement for optimizing the dyeing process. In detail, the chromosome generator 140 may assign a weight to each of the selected work commands in step S250, and may apply a roulette wheel selection method to the weight-assigned work command to select the unit genes one by one in step S260. In step S270, the chromosome generator 140 may arrange the unit genes in order in which the unit genes are selected and may determine the arrangement of the unit genes by repeating a selection so that the unit genes are arranged by a previously calculated length of chromosome, thereby completing generating of initial chromosomes.

Moreover, the chromosome generator 140 may assign a weight, set based on the degree to which a due date of an ordered work command arrives, to each of the selected work commands. In detail, the chromosome generator 140 may assign a highest weight to a work command closest to the due date and may assign a lowest weight to a work command farthest away from the due date.

Moreover, the chromosome generator 140 may assign a weight to a work command, requiring a process representing similarity and a dyeing process which is being currently performed, among the selected work commands. In detail, the chromosome generator 140 may assign a weight to each work command on the basis of a result obtained by determining similarity between a tenter process and a sub dyeing process among processes included in the dyeing process. Here, a weight assigned to a work command requiring a process representing the similarity may be set to be relatively lower than a weight assigned based on a due date, and weights may be differently assigned to the sub dyeing process and the tenter process on the basis of a result obtained by determining similarity.

In the above description, an initial chromosome generating process based on weight assignment and roulette wheel selection by the chromosome generator 140 will be described below for example. First, like J1(20), J2(14), J3(5), and J4(3) (where, J1 to J4 may each denote a work command, and a number in parenthesis may denote a weight), four work commands may be selected, and when weights “20, 14, 5, and 3” are assigned to work commands, J1 may be 48%, J2 may be 33%, J3 may be 12%, and J4 may be 7%. Here, the chromosome generator 140 may generate a random number within a range of 1 to 100 and may select and arrange unit genes of work commands within a range corresponding to the generated random number (for example, when a random number within a range of 1 to 48 is generated, a unit gene of J1 may be selected). Referring to a second row of FIG. 5, an example where unit genes are arranged in the order of J1, J2, J4, J3, . . . may be checked, and in this case, a first random number may be generated within a range of 1 to 48, a second random number may be generated within a range of 49 to 81, a third random number may be generated within a range of 94 to 100, and a fourth random number may be generated within a range of 82 to 93.

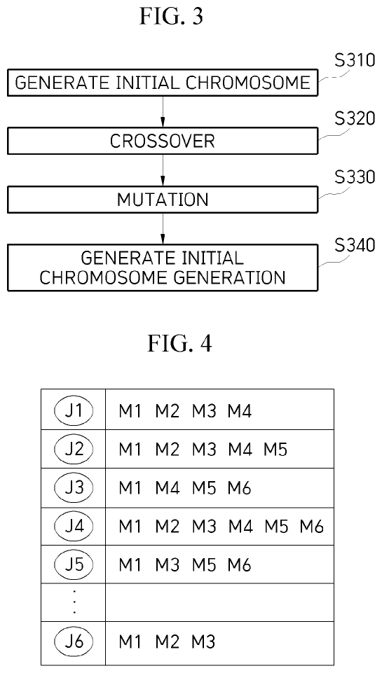

Moreover, referring to FIG. 3, the chromosome generator 140 may apply a crossover operation and a mutation operation to the generated initial chromosome to generate an initial chromosome generation in steps S310 to S340. Here, a method of applying a crossover operation and a mutation operation to the generated initial chromosome may be variously provided and may be applied as a method where highest efficiency is shown based on situations of each dyeing process.

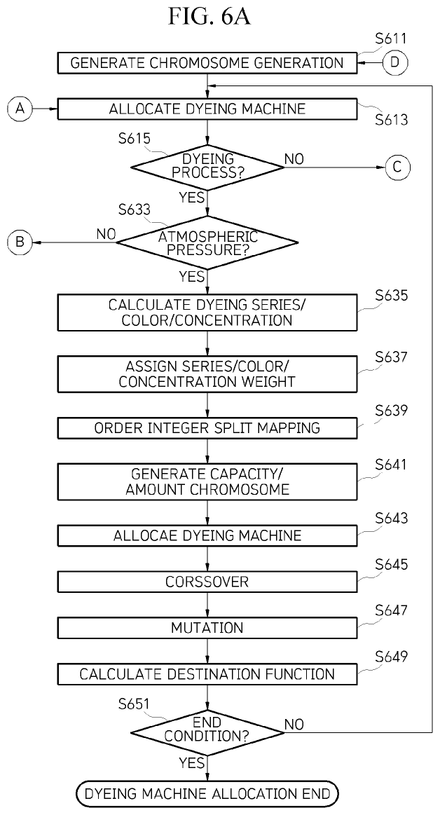

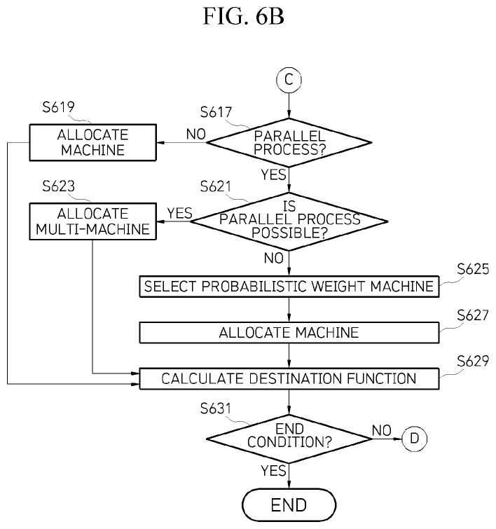

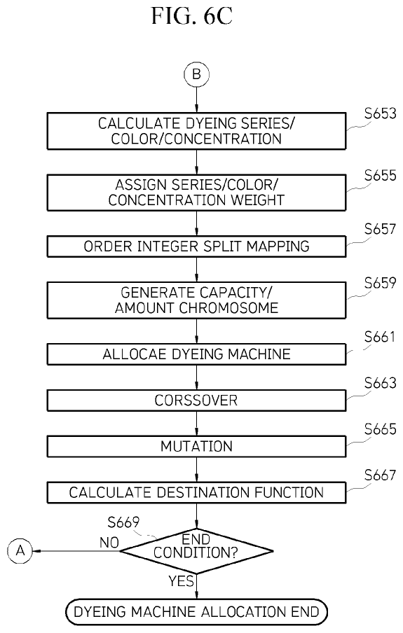

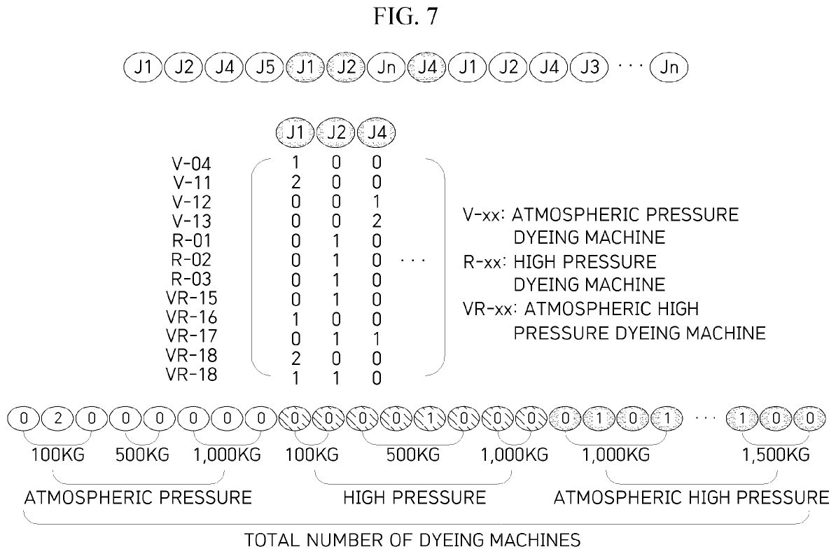

Moreover, referring to FIG. 6A to 6C, the chromosome generator 140 may allocate a machine, which is for performing a dyeing process, to each unit gene configuring an initial chromosome of the initial chromosome generation in steps S611 and S613. Here, when a process corresponding to the unit gene is not a dyeing process in step S615 (S615-N), the chromosome generator 140 may determine whether a parallel process is possible and may allocate a machine. Also, when the process corresponding to the unit gene is the dyeing process, the chromosome generator 140 may allocate a dyeing machine corresponding to a dyeing characteristic where a corresponding dyeing process is to be performed. In detail, when the process corresponding to the unit gene is not the dyeing process, the chromosome generator 140 may determine whether the parallel process is possible in step S617, and when the parallel is impossible, the chromosome generator 140 may allocate a single machine. At this time, when the parallel process is possible in step S621 (S621-Y), the chromosome generator 140 may determine whether the parallel process is possible by using a process performed in a current dyeing process, and when the parallel process is possible, the chromosome generator 140 may allocate a multi-machine in step S623.

When it is determined that the parallel process is impossible by using a process performed in the current dyeing process in step S621 (S621-N), the chromosome generator 140 may determine similarity by using information such as a series of a color, a color, a concentration, and a special situation and may assign a weight, on the basis of a previously performed process in step S625, and may allocate a machine having similarity which is the most similar in step S627.

Moreover, referring to FIG. 6A to 6C, the chromosome generator 140 may classify a work on which the dyeing process is to be performed and may calculate a series, a color, and a concentration of the classified work in steps S635 and S653, and in steps S637 and S655, the chromosome generator 140 may assign a weight to each of the dyeing machines on the basis of a result obtained by calculating similarity to a work which is being performed in a current dyeing process. In detail, the dyeing process may perform an atmospheric pressure dyeing work and a high pressure dyeing work, and thus, may include three kinds of (atmospheric pressure, high pressure, and atmospheric high pressure) dyeing machines which respectively perform dyeing works.

Therefore, a work on which the dyeing process is to be performed may be classified into an atmospheric pressure dyeing work and a high pressure dyeing work in step S633. Subsequently, like a weight assignment operation performed on a process instead of the above-described dyeing process, a weight corresponding to a dyeing machine may be assigned by calculating a series, a color, and a concentration of a chromosome on which a work is to be performed. When weight assignment is completed, a dyeing machine may be allocated based on the number of works on which a dyeing process is to be performed in steps S643 and S661, and thus, an operation of mapping the number of works to a dyeing machine may be needed by dividing the number of works by an integer (or a natural number) in steps S639 and S657.

Moreover, referring to FIG. 7, the chromosome generator 140 may generate a dyeing machine-allocation chromosome which is configured with arrangement of unit genes representing allocation information about a dyeing machine for the dyeing process and where the number of dyeing machines used in the dyeing process is set to a chromosome length. In detail, in the dyeing machine-allocation chromosome, a unit genes arrangement region representing atmospheric pressure, high pressure, and atmospheric high pressure dyeing machines may be separately designated, and a unit genes arrangement region based on the kind of a dyeing machine may be divisionally designated based on a capacity of the dyeing machine again. Also, in the present invention, the dyeing machine-allocation chromosome may be for chromosome machine allocation on a dyeing process among processes needed for a work command and may be referred to as a chromosome which is additionally generated after an initial chromosome, for configuring a genetic algorithm specialized for a dyeing process.

Moreover, referring to FIG. 7, when a dyeing machine is allocated to all unit genes corresponding to a dyeing process among unit genes configuring a certain initial chromosome, the chromosome generator 140 may generate the dyeing machine-allocation chromosome on the basis of a solution set of allocated dyeing machines. In FIG. 7, unit genes of hatched J1, J2, and J4 may each denote a dyeing process and unit genes may be arranged as a solution set of dyeing machines allocated to J1, J2, and J4, and thus, the dyeing machine-allocation chromosome may be generated.

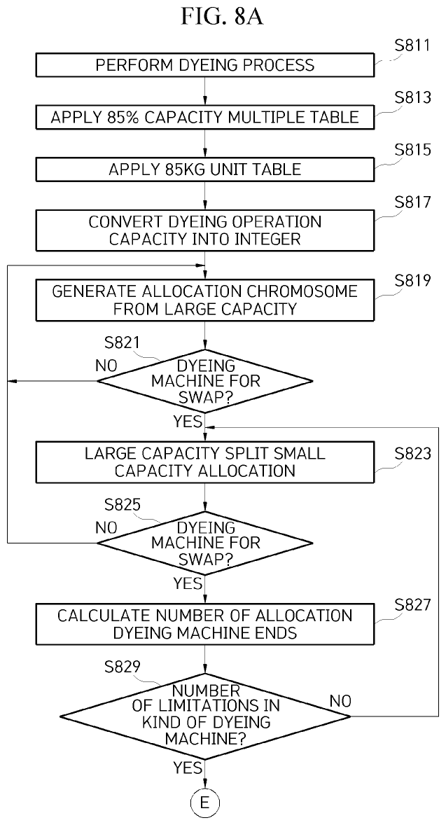

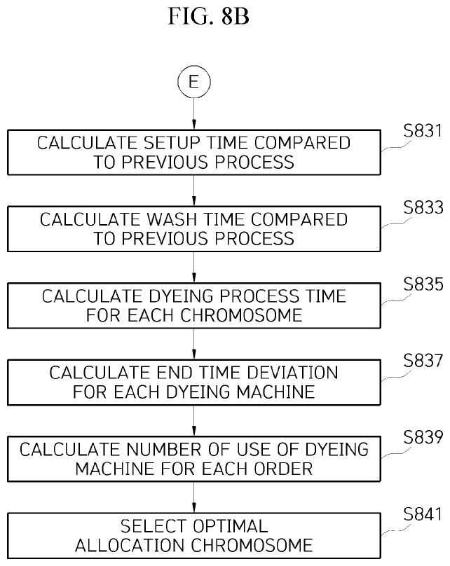

Moreover, referring to FIGS. 6 and 7, the chromosome generator 140 may apply a crossover operation and a mutation operation to the generated dyeing machine-allocation chromosome to generate dyeing machine-allocation chromosome generation in steps S645, S647, S663, and S665. Also, the chromosome generator 140 may calculate a destination function corresponding to each dyeing machine-allocation chromosome included in the generated dyeing machine-allocation chromosome generation and may calculate suitability in steps S649 and S667 and may allocate a dyeing machine on the basis of a configuration of the dyeing machine-allocation chromosome representing optimal suitability as a result of suitability calculation, thereby completing dyeing machine allocation. Here, a crossover operation and a mutation operation may be restrictively performed on the dyeing machine-allocation chromosome. For example, a work of a high pressure dyeing machine may be changed to an atmospheric pressure dyeing machine, a work of a dyeing machine corresponding to an atmospheric pressure and a work of a dyeing machine corresponding to an atmospheric high pressure may crossover therebetween, and a work of a high pressure dyeing machine may be changed to an atmospheric high pressure dyeing machine. Also, a work may be preferentially allocated to a dyeing machine having a large capacity in step S819 (see FIG. 8A), the crossover operation may apply an arithmetic operation of swapping a plurality of dyeing machines having the same capacity in step S821, and the mutation operation may apply a split operation of splitting a work allocated to a dyeing machine having a large capacity by using a dyeing machine having a small capacity in step S823.

Moreover, the chromosome generator 140 may calculate suitability on the basis of a result of calculation performed by setting, to a variable of the destination function, a dyeing process time, an end time deviation for each dyeing machine, and the number of use of a dyeing machine for each number of work based on a configuration of each dyeing machine-allocation chromosome, a setup time, and a wash time of a current process with respect to a previously performed process in a dyeing process in steps S831 to S841. FIG. 8A and 8B are flowchart illustrating a dyeing machine allocation process in more detail compared to FIG. 6A to 6C. Referring to FIG. 8A and 8B, the number of works of a dyeing process may be calculated in step S811, and the number of works may be converted into an integer so that the number of works corresponding to a capacity of 85% of each dyeing machine is introduced in steps S813, S815, and S817. Here, the integer conversion may be performed based on a capacity multiple table and a unit table shown in the following Table 1 and Table 2.

TABLE 1
capacity multiple table

| 100 Kg | 250 Kg | 500 Kg | 1000 Kg | 1500 Kg |
|---|---|---|---|---|
| 85 | 212.5 | 425 | 850 | 1275 |
| 170 | 425 | 850 | 1700 | 2550 |
| 255 | 637.5 | 1275 | 2550 | 3825 |
| 340 | 850 | 1700 | 3400 | 5100 |
| 425 | 1062.5 | 2125 | 4250 | 6375 |
| 510 | 1275 | 2550 | 5100 | 7650 |
| 595 | 1487.5 | 2975 | 5950 | 8925 |
| 680 | 1700 | 3400 | 6800 | 10200 |
| 765 | 1912.5 | 3825 | 7650 | 11475 |
| 850 | 2125 | 4250 | 8500 | 12750 |

TABLE 2
85 kg unit table

| 100 Kg | 250 Kg | 500 Kg | 1000 Kg | 1500 Kg |
|---|---|---|---|---|
| 85 | 212.5 | 425 | 850 | 1275 |
| 170 | 297.5 | 510 | 935 | 1360 |
| 255 | 382.5 | 595 | 1020 | 1445 |
| 340 | 467.5 | 680 | 1105 | 1530 |
| 425 | 552.5 | 765 | 1190 | 1615 |
| 510 | 637.5 | 850 | 1275 | 1700 |
| 595 | 722.5 | 935 | 1360 | 1785 |
| 680 | 807.5 | 1020 | 1445 | 1870 |
| 765 | 892.5 | 1105 | 1530 | 1955 |
| 850 | 977.5 | 1190 | 1615 | 2040 |
| 935 | 1062.5 | 1275 | 1700 | 2125 |
| 1020 | 1147.5 | 1360 | 1785 | 2210 |
| 1105 | 1232.5 | 1445 | 1870 | 2295 |
| 1190 | 1317.5 | 1530 | 1955 | 2380 |
| 1275 | 1402.5 | 1615 | 2040 | 2465 |
| 1360 | 1487.5 | 1700 | 2125 | 2550 |

Subsequently, as described above, a crossover operation and a mutation operation may be applied, an optimal dyeing machine-allocation chromosome may be generated based on suitability obtained by calculating a set destination function, and variables used for the destination function may be used to calculate suitability as in the following Equation 1 and Equation 2.

Minimize(1+k)^{WashCount-1}(1+p)ⁿ⁻¹ (Σ(ET−MTᵢ)+1) [Equation 1]
k: penalty constant based on use of water and drug (k>0)
p: error rate (0<p<1)
n: number of dyeing machines used in dyeing process
MTᵢ: work completion time of each dyeing machine
ET: largest value in MTᵢ, work completion time of dyeing process

$${SetupTime}_{i} = \{ \begin{matrix}0 if {Af}_{n} , {Co}_{n} , {Cb}_{n} is equal {Af}_{w} , {Co}_{w} , {Cb}_{w} \\ 2 if {Af}_{n} , {Co}_{n} is equal {Af}_{w} \\ 3 if {Af}_{n} < {Af}_{w} \\ 4 if {Af}_{n} \geq {Af}_{w}\end{matrix} \qquad [ Equation 2 ]$$

i: index of dyeing machine
n: allocated work
w: work allocated to dyeing machine
Af: series of color
Co: color concentration
Cb: color combination

The process controller 150 may perform each process on the basis of a configuration of a chromosome representing optimal suitability among the generated initial chromosomes to perform the selected work command. In detail, when a machine allocation operation and a suitability calculation operation on each of a plurality of initial chromosomes configuring the generated initial chromosome generation according to an embodiment of the present invention are completed, a process scheduling operation may end, and the process controller 150 may perform a dyeing process on the basis of a configuration of a chromosome having optimal suitability, thereby implementing production having maximum efficiency.

A dynamic scheduling system according to the present invention may be applied to a manufacturing process including a process similar to a dyeing process. According to an aspect to the present invention, a dynamic scheduling system for a manufacturing process of optimizing performing of an ordered work command through scheduling on each process of the manufacturing process includes: a data collector configured to collect order data associated with an ordered work command in the manufacturing process and process data associated with each process included in the manufacturing process; a work time calculator configured to calculate a work time of each process on the basis of the collected order data and process data; a work command determiner configured to select a work command, which is to be performed, from among ordered work commands on the basis of the calculated work time; a chromosome generator configured to set each process, needed for performing the selected work command, to unit genes and combine the unit genes, set to correspond to each process, to generate a chromosome having optimal suitability for the manufacturing process; and a process controller configured to perform each process on the basis of a configuration of a chromosome representing optimal suitability among the generated initial chromosomes to perform the selected work command.

Here, the chromosome generator may assign a weight to each of the selected work commands and may generate an initial chromosome configured with arrangement of unit genes obtained based on the assigned weight.

Moreover, the chromosome generator may allocate a machine, which is for performing a dyeing process, to each unit gene configuring the initial chromosome and may allocate a machine on the basis of the kind of a process corresponding to a unit gene configuring the initial chromosome.

Referring to an embodiment of the dynamic scheduling system 100 for a dyeing process using a genetic algorithm described above, a dynamic scheduling system for a manufacturing process may apply fundamentally the same genetic algorithm to a process other than a process associated with a sub dyeing process. For example, the dynamic scheduling system for the manufacturing process may assign a weight to the selected work command by using the chromosome generator, on the basis of the degree to which a due date arrives and similarity to a currently performed dyeing process.

In the dynamic scheduling system and method of a dyeing process using a genetic algorithm according to the embodiments of the present invention, a total process may be optimized through integrated scheduling of a plurality of processes included in a dyeing process.

The dynamic scheduling system and method of a dyeing process using a genetic algorithm according to the embodiments of the present invention may perform more accurate process scheduling even without depending on a process scheduling manager of a conventional dyeing process.

The dynamic scheduling system and method of a dyeing process using a genetic algorithm according to the embodiments of the present invention may perform scheduling of module processes such as a tenter process, a sub dyeing process, and a total process of a dyeing process, thereby realizing optimal production efficiency.

The dynamic scheduling system and method of a dyeing process using a genetic algorithm according to the embodiments of the present invention may obtain an optimal effect of a process based on various variable influences such as a capacity of a dyeing machine, the possibility of a parallel process, and a parameter value of a previously performed process.

A number of exemplary embodiments have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. A dynamic scheduling method of a dyeing process using a genetic algorithm which optimizes performing of an ordered work command through scheduling on each process of the dyeing process, the dynamic scheduling method comprising:
selecting a work command, which is to be performed, from among ordered work command in the dyeing process;
setting each process, needed for performing the selected work command, to a unit gene and combining unit genes, set to correspond to each process, to generate an initial chromosome;
allocating a machine for performing a dyeing process on each unit gene configuring the initial chromosome; and
calculating suitability for the initial chromosome to which the machine is allocated.

2. The dynamic scheduling method of claim 1, wherein the generating of the chromosome comprises calculating a length of the chromosome configured by a combination of the unit genes.

3. The dynamic scheduling method of claim 2, wherein the calculating of the length of the chromosome comprises determining, as the length of the chromosome, a value which is a sum of unit genes configuring the selected work command.

4. The dynamic scheduling method of claim 1, wherein the generating of the chromosome comprises:
assigning a weight to each of the selected work commands; and
applying a roulette wheel selection method to the weight-assigned work command to generate the initial chromosome including the unit gene.

5. The dynamic scheduling method of claim 4, wherein the assigning of the weight to each of the selected work commands comprises assigning a weight set based on the degree to which a due date of a work command arrives.

6. The dynamic scheduling method of claim 4, wherein the assigning of the weight to each of the selected work commands comprises assigning a weight to each of the selected work commands on the basis of a result obtained by determining similarity between a tenter process and a sub dyeing process in a currently performed dyeing process.

7. The dynamic scheduling method of claim 4, wherein the generating of the chromosome comprises applying a crossover operation and a mutation operation to the generated initial chromosome to generate initial chromosome generation.

8. The dynamic scheduling method of claim 1, wherein the allocating of the machine comprises:
when a process corresponding to the unit gene is not a sub dyeing process, allocating a machine by determining whether a parallel process is possible; and
when the process corresponding to the unit gene is the sub dyeing process, allocating a dyeing machine corresponding a dyeing characteristic where a corresponding sub dyeing process is to be performed.

9. The dynamic scheduling method of claim 8, wherein the allocating of the dyeing machine comprises generating a dyeing machine-allocation chromosome which is configured with arrangement of unit genes representing allocation information about a dyeing machine for the sub dyeing process and where the number of dyeing machines used in the sub dyeing process is set to a chromosome length.

10. The dynamic scheduling method of claim 8, wherein the allocating of the dyeing machine comprises:
classifying a work on which the sub dyeing process is to be performed;
calculating a series, a color, and a concentration of the classified work and assigning a weight to each of the dyeing machines on the basis of a result obtained by calculating similarity to a work which is being performed in a current sub dyeing process; and
allocating the dyeing machine on the basis the weight assigned to the dyeing machine and the number of works of the classified work.

11. The dynamic scheduling method of claim 9, wherein the allocating of the dyeing machine comprises:
applying a crossover operation and a mutation operation to the generated dyeing machine-allocation chromosome to generate dyeing machine-allocation chromosome generation;
calculating a destination function corresponding to each dyeing machine-allocation chromosome included in the generated dyeing machine-allocation chromosome generation and calculating suitability; and
allocating a dyeing machine on the basis of a configuration of the dyeing machine-allocation chromosome representing optimal suitability as a result of the suitability calculation.

12. A dynamic scheduling system for a dyeing process using a genetic algorithm which optimizes performing of an ordered work command through scheduling on each process of the dyeing process, the dynamic scheduling system comprising:
a data collector configured to collect order data associated with the ordered work command and process data associated with each process included in a dyeing process;
a work time calculator configured to calculate a work time of each process on the basis of the collected order data and process data;
a work command determiner configured to select a work command, which is to be performed, from among ordered work commands on the basis of the calculated work time;
a chromosome generator configured to generate initial chromosomes, set to unit genes for configuring a chromosome in each process needed for performing the selected work command, and a dyeing machine-allocation chromosome for allocating a dyeing machine for unit chromosomes corresponding to a sub dyeing process of each process; and
a process controller configured to perform each process on the basis of a configuration of a chromosome representing optimal suitability among the generated initial chromosomes to perform the selected work command.

13. The dynamic scheduling system of claim 12, wherein the chromosome generator assigns a weight to each of the selected work commands and generates an initial chromosome configured with arrangement of unit genes obtained based on the assigned weight.

14. The dynamic scheduling system of claim 13, wherein the chromosome generator assigns a weight to the selected work command on the basis of the degree to which a due date arrives and similarity to a currently performed dyeing process.

15. The dynamic scheduling system of claim 12, wherein the chromosome generator allocates a machine, which is for performing a dyeing process, to each unit gene configuring the initial chromosome and allocates a machine on the basis of the kind of a process corresponding to a unit gene configuring the initial chromosome.

16. The dynamic scheduling system of claim 12, wherein the chromosome generator generates a dyeing machine-allocation chromosome which is configured with arrangement of unit genes representing allocation information about a dyeing machine used in a sub dyeing process of each process and where the number of dyeing machines used in the sub dyeing process is set to a chromosome length.

17. The dynamic scheduling system of claim 12, wherein the chromosome generator
calculates suitability for the dyeing machine-allocation chromosome to complete dyeing machine allocation on a unit gene corresponding to the sub dyeing process, and
calculates suitability of an initial chromosome where machine allocation is completed on each process.

18. A dynamic scheduling system for a manufacturing process of optimizing performing of an ordered work command through scheduling on each process of the manufacturing process, the dynamic scheduling system comprising:
a data collector configured to collect order data associated with an ordered work command in the manufacturing process and process data associated with each process included in the manufacturing process;
a work time calculator configured to calculate a work time of each process on the basis of the collected order data and process data;
a work command determiner configured to select a work command, which is to be performed, from among ordered work commands on the basis of the calculated work time;
a chromosome generator configured to set each process, needed for performing the selected work command, to unit genes and combine the unit genes, set to correspond to each process, to generate a chromosome having optimal suitability for the manufacturing process; and
a process controller configured to perform each process on the basis of a configuration of a chromosome representing optimal suitability among the generated initial chromosomes to perform the selected work command.

19. The dynamic scheduling system of claim 18, wherein the chromosome generator assigns a weight to each of the selected work commands and generates an initial chromosome configured with arrangement of unit genes obtained based on the assigned weight.

20. The dynamic scheduling system of claim 18, wherein the chromosome generator allocates a machine, which is for performing a dyeing process, to each unit gene configuring the initial chromosome and allocates a machine on the basis of the kind of a process corresponding to a unit gene configuring the initial chromosome.
